(19) United States
(12) Patent Application Publication
(10) Pub. No.: US 2025/0057731 A1
(43) Pub. Date: Feb. 20, 2025

(54) SUPPORTIVE PHASE SYSTEM FOR PRODUCING ANTIBACTERIAL AND REGENERATIVE DENTAL COMPOSITE FILLING MATERIALS

(71) Applicant: YILDIZ TEKNIK UNIVERSITESI, Istanbul (TR)

(72) Inventors: Afife Binnaz HAZAR, Istanbul (TR); Aysu AYDINOGLU, Istanbul (TR); Kadir SAGIR, Istanbul (TR)

(73) Assignee: YILDIZ TEKNIK UNIVERSITESI, Istanbul (TR)

(21) Appl. No.: 18/722,598

(22) PCT Filed: Dec. 5, 2022
(86) PCT No.: PCT/TR2022/051405

§ 371 Date: Jun. 21, 2024

(30) Foreign Application Priority Data
Dec. 27, 2021 (TR) 2021/021083

(51) Int. Cl. A61K 6/75; A61K 6/62; A61K 6/78; A61K 6/887

(52) U.S. Cl. CPC A61K 6/75; A61K 6/62; A61K 6/78; A61K 6/887

(57) ABSTRACT

An acrylic dental composite filling material and a production method thereof are provided. The acrylic dental composite filling material includes a light-cured and polymerizable organic compound, a photoinitiator, and a supportive phase system, where the light-cured and polymerizable organic compound is a mixture of BisGMA and TEGDMA, the photoinitiator is at least one of CQ, diphenyl(2,4,6-trimethylbenzoyl) phosphine oxide, 1-phenyl-1,2-propanedione, and 4-EDMAB.

### CROSS REFERENCE TO THE RELATED APPLICATIONS

This application is the national International Application No. PCT/TR2022/051405, filed on Dec. 5, 2022, which is based upon and claims priority to Turkish Patent Application No. 2021/021083, filed on Dec. 27, 2021, the entire contents of which are incorporated herein by reference.

### TECHNICAL FIELD

The invention relates to the light-cured and polymerizable restorative acrylic dental composite filling materials and a new supportive phase system for producing the said dental composites, and to the production of a new acrylic dental composite filling material for the related technical field.

### BACKGROUND

Resin-based composite (RBC) materials were first reported by Bowen in 1958. On the other hand, the commercial use of resin-based composites was made possible by the author's patent titled “a vinyl-silane treated fused silica and binder” in 1962. The concept of chemically cured RBCs became a concept only after they were introduced to the dental market in 1970. On the other hand, although the applications performed without abrasive in large-scale restorations are limited, these materials are frequently preferred in Grade I and Grade II Restorations.

In 1974, the acquisition of the patent named “A method of repairing teeth using a composition which was curable by visible light” by Dart and the development of “total-etch” adhesives in the 1980s paved the way for these materials by supporting the clinical use of light-cured RBCs in Grade I and Grade II restorations.

Due to the increasing expectations in aesthetic dentistry in recent years, the development of clinically long-lasting resin composites with advanced physical and mechanical properties, providing aesthetic needs, has been inevitable for use in direct restorations. One of the most important developments in this field is the use of nanostructured particles in the structure of traditional resins by combining them with nanocrystals.

The history of existing resin monomers is based on the discovery of a new acid called “acrylic acid” by the German chemist J. Redtenbacher. In the 1900s, besides the synthesis of methacrylic acid and many ester derivatives, methyl methacrylate polymers were also produced with the polymerization technique. In the last years of the 1930s, polymethyl methacrylate first entered the field of dentistry as denture pedestal resins and a few years later, it was used as indirect filling materials. With the discovery of benzoyl peroxide-tertiary amine redox initiator-accelerator systems in Germany during World War II, polymerization of methyl methacrylate at room temperature could be carried out, and thus these polymeric structures could be used as direct filling materials. On the other hand, these materials have failed to meet the necessary clinical expectations.

Observing the inadequate properties of methyl methacrylate resins, American dentist R. L. Bowen developed other synthetic resins for use in dental filling materials. In this context, he carried out studies on epoxy resins that can be polymerized at room temperature. Although epoxy resins exhibit good aesthetic properties in the oral cavity, the slowing of the hardening phase has prevented their use directly as filling materials.

Depending on the problems encountered in epoxy resins, Bowen conducted studies on a new monomer system, 2,2-bis [4-(2-hydroxy-3-methacryloxypropoxy)phenyl]propane-bisphenol A-glycidyl methacrylate (BisGMA) structure in 1956. This monomer system has been found to be highly superior compared to methyl methacrylate systems due to its high molecular weight, chemical structure, low volatility, low polymerization shrinkage, and rapid hardening properties.

BisGMA has found wide use in commercial dental resin composites. On the other hand, the color stability of the monomer is insufficient, highly viscous, and could not be purified by methods such as distillation and crystallization. In order to prevent this problem, Bowen has conducted studies on isomeric crystalline dimethacrylates, which exhibit eutectic formations at room temperature and are liquid, which is a new monomeric system. In this context, three aromatic diesters of phthalic (P), isophthalic (I), and terephthalic (T) acids synthesized the bis(2-methacryloxyethyl)-P/I/T ester monomer and purified it by recrystallization method. It has been determined that the mechanical properties of the composites produced from these monomers are equivalent to BisGMA and the polymerization shrinkage values meet the expected property. On the other hand, these monomer systems could not provide the expected color stability in vivo.

Depending on their polar structure, dimethacrylate resins tend to adsorb water in the oral environment and exhibit hygroscopic expansion. Although this expansion has some advantages, it causes various disadvantages such as decreased mechanical strength and wearing resistance in the long term. For this reason, hydrophobic monomer systems obtained by removing the hydroxyl groups in the BisGMA chains have been developed to minimize the water retention capacity of the resin. However, these systems could not meet the desired mechanical properties.

Since the expected properties of hydrophobic monomers were not met, various studies were conducted to develop fluorocarbon-containing polymers with low surface energy and high hydrophobic properties. In this context, polyfluoro monomethacrylate and octafluoropentyl methacrylate monomer systems were used in resin composites. Although these composite structures provide the expected hydrophobic properties against water, they have not provided sufficient physical and mechanical properties as well as exhibiting fairly high polymerization shrinkage.

Urethane dimethacrylates (UDM), another monomer system used in composite resins, were first synthesized from hydroxyalkyl methacrylates and diisocyanates. Although this monomer has a similar molecular weight to BisGMA, it has a lower viscosity value. The higher percentage of monomer conversion of composite systems produced using UDM monomers compared to BisGMA systems ensures that their biocompatibility is also higher. These systems find a place of use as an alternative to BisGMA monomer in commercial resin composites, depending on their economical nature.

Polymerization shrinkage is the most common problem in composite resins. The approximate volume shrinkage of BisGMA-based polymers is around 5% and this value can be reduced by increasing the loading amount of the supportive phase systems. Since polymerization shrinkage is one of the important parameters affecting the duration of use of the composite, various studies have been conducted on non-shrinkage and polymerized bicyclic compounds by the cycle opening polymerization technique. Bailey stated that various bicyclic monomers and unsaturated dicetels containing spiro orthocarbonates, spiro orthocarbonates, bicyclic keto lactones, and trioxabicyclo octanes exhibit double ring polymerization without shrinkage and/or expansion. On the other hand, at the end of the reaction, it was determined that there was a large number of monomers that did not add to the polymeric structure in the environment.

Developments in organic resin structure were followed by the production of polyacid-modified composites known as “compomers” in 1994. These composite materials are obtained by embedding supportive phase systems such as calcium-aluminum-fluorosilicate glasses in polymer resin. Compomers are dental materials that combine the aesthetic properties of conventional composites and the fluoride release and adhesion properties of glass ionomer cement. In contrast, compomers are separated from glass ionomer cement in two ways: firstly, glass particles are partially silanized to enable binding with resin, and secondly, the polymeric structure is formed as a result of radical polymerization reactions following the activation of monomers by light.

In 1984, Schmidt first synthesized the organic-inorganic polymeric hybrid structures called “ormosils (organically modified silicates)” and later “ormocer (organically modified ceramics)”. In ormocers, organic and inorganic compounds come together on a nanoscopic, that is, molecular scale. For this reason, although these materials have the properties of organic and inorganic components in their structures, they exhibit unique features whose cause has not yet been understood [9]. Ormocers started to be used in the field of dentistry in 1998, and especially in recent years, ormocers have found commercial use in restorative material applications.

One of the technological developments in dental commercial materials in recent years is the use of silorane-based organic resin monomers. The silorane monomer is named after the siloxane and oxirane molecules that make up its structure. Silorane monomers developed within 3M ESPE to reduce polymerization shrinkage, which is predicted to be an alternative to methacrylate-based monomers, and to obtain composite materials that maintain appropriate mechanical strength, have been able to find commercial application in a short time, depending on whether they meet the desired clinical expectation.

In addition to reducing the polymerization shrinkage, another feature that is desired to be brought into the monomer systems is the antibacterial effect. In this context, instead of adding fluoride-releasing compounds to the structure, monomers exhibiting antibacterial properties were produced. In this context, the most promising monomer is methacrylyl-dodecylpyridinium bromide (MDPB) produced by the reaction of the antibacterial agent dodecylpyridinium bromide and methacrylate group. Various studies have determined that composite systems produced as a result of the copolymerization of conventional dental monomers and methacrylyl-dodecylpyridinium (MDP) have an inhibitory effect on bacterial formation on the surface. On the other hand, in today's commercially produced composite systems, the antibacterial effect is carried out through supportive phase particles that release fluoride instead of being provided through such monomer systems.

In the 1960s, three basic materials were used in the structure of the first composite resin: monomer, silane-treated supportive phase, and initiator. The supportive phase used by Bowen in 1963 consists of ground quartz particles with an average size of 8-12 μm (8000-12000 nm). Due to the limitations of macrophilic composites in aesthetic restorations (such as problems with surface polishing), minifill composites were developed in the 1970s. Supportive phase systems produced by pyrogenic methods increased the polishing ability by making it possible to load composites at a maximum of 55% but significantly reduced the mechanical strength.

It was only possible to test the supportive phase systems as a mixture in 1980 and 1990. These restorative materials containing hybrid supportive phase systems with particle sizes of 600-2,000 nm are commercialized as hybrid, microhybrid and condensed (whisker structure) composites. With these products, the mechanical strength has been significantly increased, but the polishability is still limited. In these products, the maximum loading rate reached 70-77% by weight. Nevertheless, the particle size values of traditional composites could not provide sufficient harmony with the hydroxyapatite crystals, dentin tubules, and enamel rods in the natural structure of the tooth. For this reason, the potential to provide the necessary adhesion between the macroscopic restorative material and the nanoscopic (1-10 nm) tooth structure could not be obtained.

Since it is not possible to produce supportive phases below 100 nm by grinding method, nanotechnological methods have been an innovative technique in this field by providing features such as the use of supportive phase production, controlled crystal growth, and the homogeneity of the final product in terms of structure and size. At the beginning of the current century, Filtek Supreme (3MESPE, St. Paul, USA) has been the commercial turning point of nanotechnology applications in operative dentistry. In this context, the composite resin produced includes 78.5% by weight of clumped (aggregate) zirconia/silica clusters with the main particle size of 5-20 nm and silica-based supportive phase systems with a particle size of 20 nm without clumping.

In recent years, composite systems containing microhybrid and nanophilic composites have been commercialized. This novel composition has enabled the loading rates of the supportive phase systems to be increased by up to 87% by weight due to the formation of filler cavities between the larger particles and the smaller particles of the supportive phase content.

The curing techniques used for the polymerization of composites have also evolved, depending on the development of the supportive phase and monomer systems. As previously mentioned, the first composite filling materials used in dentistry were polymerized with redox reactions at room temperature. Polymerization reactions of these products, which were packaged as two separate materials, started with the mixing of the products and required a long period of 8 minutes to complete the polymerization properly.

Depending on the length of the polymerization period, photopolymerizable composite systems (Nuva; Dentsplay/Caulk) were developed in the late 1970s. Such a polymerization method provided the dentist with the advantage of curing quickly and making the desired contour after placing the product. In these systems, quartz lamps with a wavelength of 354 nm were used as UV light sources at first, and the polymerization reaction proceeds through the free radical formation. Although this system is advantageous at first, various problems such as polymerization not being completely realized and depletion of the light source very quickly have been encountered in cases where the intensive application is made in the following periods. For this reason, visible light energy in the wavelength range of 400-500 nm was started to be applied instead of ultraviolet light for photopolymerization in the following periods. In visible light systems, quartz-tungsten-halogen light sources were first used. The most commonly used photoinitiator system in these systems is camphorquinone (CQ). The polymerization time of a 2 mm thick restoration with traditional quartz-tungsten-halogen light sources takes approximately 40-60 seconds. Although these systems are more advantageous for polymerization compared to self-curing systems, the search for new methods has continued because the energy of light on the composite surface is more intense than in the lower regions and the light penetrates the lower regions to a lower intensity.

With the development of laser technologies that provide high light intensity in the energy band range required for photopolymerization in dental materials, dentistry applications have also improved. “Argon laser”, which provides high energy output at 448 nm wavelength, has provided various advantages such as rapid polymerization in commercial dental restorative materials.

Another system developed to shorten the polymerization time is “Plasma Arc Units”. In this context, short arc systems provided by the use of xenon light sources are named plasma arc light sources. This unit consists of spark and fluid gas systems produced by applying high energy potential between two tunnels. The system operates at 400-500 nm wavelength and polymerization takes place in less than 1 second. On the other hand, some researchers suggest that the characteristics of the final product will not be of the desired quality since polymerization takes place in such a short time.

In order to keep quartz-tungsten-halogen lamps on the market, manufacturers have developed the existing system by providing “high energy” output and short application time similar to plasma arc systems. On the other hand, there were concerns about polymerization shrinkage due to the development of fast curing conditions.

The developments observed in light emitting diode (LED) technology have been inevitable for the use of these light sources in the dentistry industry. The absorption range of the blue LED light sources corresponds to the wavelength range of the dental photoinitiator systems. The main advantages of this light source can be listed as its portability, minimum maintenance requirement, long service life, and absorption only at the wavelength required for the activation of the photo starter. Another curing method used especially in cement technologies is the dual-cure method. In the chemical curing method, this problem has been tried to be solved by the light curing method since the excess initiator remaining in the environment has to be removed from the environment and waited for a long time until the product is fully settled. On the other hand, in light curing, complete polymerization cannot be achieved since the light is not sufficiently intense in the depths. In order to prevent these problems, dual-cure resin cement systems have been developed by using both chemical and photoinitiator systems in the products.

One of the most important cases in composite restorations is the monomer conversion percentage. One of the simplest methods to achieve polymerization is to apply heat. Heat reduces monomer viscosity, allowing free radicals to better diffuse into the monomer, and a higher percentage of monomer conversion is observed. Based on this principle, the “post-cure heating” method has been developed for photoinitiator composite systems. In this method, the composite is first cured with a conventional light source and photopolymerized and then heat is applied. This method is preferred in glass ionomer systems rather than composite filling materials.

Resin-based composite restorations that started with methyl methacrylate resin compositions have come a long way in terms of organic resin, inorganic phase, and curing techniques. On the other hand, resin-based composite filling materials do not have a structure that can mimic the natural tooth structure and do not have sufficient features that can meet clinically necessary expectations. Therefore, considering the clinical expectation, research on the development of the properties of composite filling materials continues at an increasing rate.

In 2014, Martin et al. carried out studies on the synthesis of a urethane multimetacrylate-based monomer system, which may be an alternative to BisGMA-based composites that cause problems such as low monomer conversion and high volume shrinkage. In this context, they produced urethane-multimetacrylate monomer by using methacryloyloxypropylphenylmethane (BMPM) and urethane-methacryloyloxyethyl (UME) initial monomers. By loading silica-supportive phase particles into this monomer system, they determined that the composite they produced showed lower polymerization shrinkage and higher bending strength compared to conventional composites and that the physical, chemical, and mechanical properties of the composite had a certain balance.

Liu et al. drew attention to the problems of homogeneous dispersion of Ag nanocrystals used in dental composites in organic resin to prevent secondary caries formation and loaded silver particles into composite systems by modifying them with organic agents. In this context, Ag coated the nanoparticles with oleic acid and examined the mechanical and antibacterial properties of dental resin composites. They determined that modified silver nanoparticles significantly improved the mechanical and antibacterial properties of the composite, such as bending strength, elasticity modulus, and compressive strength, compared to unmodified particles.

He et al. conducted studies on the synthesis of antibacterial and radiopaque dimethacrylate monomers for use in dental resin composites. In this context, they synthesized tertiary ammonium dimethacrylate compounds such as N,N-bis [2-(3-(methacryloyloxy) propanamido)ethyl]-N-methyldodecyl ammonium iodide (QADMAI-12), N,N-bis [2-(3-(methacryloyloxy) propanamido)ethyl]-N-methylhexadecyl-ammonium iodide (QADMAI-16) and N,N-bis [2-(3-(methacryloyloxy) propanamido)ethyl]-N-methyloctadesethyl ammonium iodide (QADMAI-18). They determined that the monomer conversion percentages of the produced composite were better compared to conventional composites as well as antibacterial and radiopacity properties. On the other hand, they observed that the flexural strength and elasticity modulus values of the composites were lower than traditional composites.

Liu et al. conducted studies on a light-curable biocidal dental resin composite. They have loaded the supportive phase system produced by combining poly(BisGMA)-graft-silanized whisker hydroxyapatite (PGSHW) and silanized-silica (s-SiO₂) nanoparticles into bisphenol-A glycidyl methacrylate (BisGMA)/triethylene glycol dimethacrylate (TEGDMA) based dental resin. They determined that the composites containing the PGSHW/s-SiO₂ hybrid supportive phase system they produced significantly improved the bending strength, elasticity modulus, compressive strength and toughness, and monomer conversion percentage values of the material compared to the composites containing hydroxyapatite nanoparticles. In addition, they have shown that the composite produced by in vitro bioactivity tests can form apatite.

Wu et al. developed a self-repairing composite containing dimethylaminohexadodecyl methacrylate (DMAHDM) to provide antibacterial function and nano-sized amorphous calcium phosphate (NACP) for remineralization to find solutions to problems such as cracking and secondary caries occurring in composite restorations. As a result of their study, they stated that the composite structure, which was produced for the first time in the literature, had the capability of crack repair, antibacterial effect, and remineralization after crack formation.

Like Wu et al., Chan et al. conducted studies on composite systems with antimicrobial effects. They have loaded nano-sized amorphous calcium phosphate ceramics and dimethylaminododecyl methacrylate (DMADDM) monomeric structures into organic resin systems of conventional composites. As a result of their study, they determined that Ca and Pion release increased and DMADDM monomer inhibited bacterial growth by showing an antimicrobial effect in connection with the pH decreasing as a result of bacterial growth and suggested that it may be appropriate to use these materials as supportive phase systems in composites.

Like Chan et al., Zhang et al. used dimethylaminododecyl methacrylate (DMADDM) with an antimicrobial effect, amorphous calcium phosphate nanoparticles as well as silver nanoparticles (NAg) as a supportive phase system and determined that the antimicrobial effect was longer compared to other composites.

Zhou et al. developed a new antimicrobial monomer system and loaded amorphous calcium phosphate nanoparticles into this system to prevent the formation of biofilm observed on the composite structure and secondary caries that occur accordingly. As antimicrobial monomers, they synthesized dimethylaminododecyl methacrylate (DMADDM) monomers with 12 carbon chains, similar to the studies of Chan and Zhang, using dimethylaminohexane methacrylate (DMAHM) monomers with 6 carbon chains, and loaded amorphous calcium phosphate nanoparticles (nACP) produced by spray dryer technique as the supportive phase to this structure. As a result of their study, they determined that the DMADDM monomer showed more antimicrobial effects compared to the DMAHM monomer. They determined that DMADDM-NACP nanocomposites had similar strength values compared to systems where tertiary ammonium dimethacrylates were used, whereas biofilm formation on the composite was reduced by only 5%. In light of their findings, they suggested that the length of the carbon chain of the monomer was highly effective in antimicrobial activity.

Jan et al. focused on polymerization shrinkage, which is one of the most common problems in dental restorations, and aimed to reduce the polymerization shrinkage of the composite structure and improve its hardness values by modifying dimethacrylate monomers with diisocyanate side groups. As a result of the studies, they determined that polymerization shrinkage can be reduced and the surface hardness of the composite can be increased depending on the chain length with the use of diisocyanate side groups.

Khan et al. carried out studies to improve the bonding between the filling material and the tooth interface and to provide fluoride release. In this context, nano fluorapatite (nFA) particles produced by the sol-gel method were bonded to organic resin consisting of urethane monomeric structures through diisocyanate side chains. They determined that the produced composite showed better bonding to the tooth structure compared to traditional composites and that fluoride was released in the long term and suggested that this composite structure could be used as filling material.

Liu et al. examined the morphology, loading, and mechanical properties of the composite by adding silanized hydroxyapatite (DK-sHA) particles with a morphology similar to that of sea urchin to composites containing and without BisGMA/TEGDMA organic resin structures containing silica nanoparticles. They determined that the mechanical properties of the composite could be improved by loading the DK-sHA support phase to the silica-free composite structures at 5% and 10% by weight and that the composite's elasticity modulus and microhardness values increased at loading levels of 20% to 30%, but the strength did not increase more. They determined that DK-sHA was embedded in the resin and distributed homogeneously in the composite compared to silanized amorphous hydroxyapatite and whisker hydroxyapatite. It has been determined that the strength and elasticity modulus values of the composite can be significantly improved if they are loaded on composite systems including silica particles.

Tauböck et al. examined the effect of alkaline bioactive glass nanoparticles (SiO₂—Na₂O—CaO—P₂O₅—Bi₂O₃) loaded into dental resin on the properties of the composite. They determined that 20% loading did not have an effect on microhardness, but significantly increased the monomer conversion percentage.

Hojati et al. loaded ZnO nanoparticles on the composite material to improve the antimicrobial effect of dental restorative materials and evaluated the physical and mechanical properties of the material with the antimicrobial effect of the composite on Streptococcus mutans bacteria. They determined that the bacterial development decreased significantly with the increasing loading of ZnO nanoparticles and that the flexural strength, compressive modulus, and monomer transformation values did not change compared to traditional composite systems.

Jan et al. modified the Bis-GMA monomers and added toluene 2,4-diisocyanate (TDI) and 1,6-hexamethylene diisocyanate (HDI) functional side groups to this monomer system to find a solution to the polymerization shrinkage problem of dental composites. They determined that resin containing high functional side groups and modified with TDI showed less cytotoxic effect than resin modified with HDI. In addition, they suggested that resin modified with TDI caused less toxic effects compared to BisGMA monomers due to the compression of toxic resin monomers in the structure.

Teeth are organs involved in many systems such as aesthetic appearance, pronunciation, and digestion. Diseases in the teeth are described as problems that directly affect the quality of life of individuals. Health problems that occur in teeth, especially in caries, can negatively affect the fulfillment of life functions, as well as affect the economy of individuals and countries. Caries are one of the most common diseases in the dental and oral environment from the past to the present. According to the data of the World Health Organization, caries is described as the most common non-communicable disease globally after the common cold. In the treatment of caries, the degraded tissue is removed from the structure and replaced with filling materials that can perform the functions of the tooth such as biting and chewing. Although various filling materials such as amalgam, glass ionomer, and composite have been used from past to present, the biggest problems seen in these materials are the deterioration of the connection between the composite filling and the tooth due to secondary caries. For this reason, the production of composites that will enable the prevention of secondary caries is one of the most investigated issues. High release and high loading rate consisting of composites reinforced with various structures such as AlF₃, Ag, MgO, ZnO, chlorhexidine (CHX), MDPB, and glass ceramic used for this purpose reduce the mechanical properties of the composite. Moreover, intervention is required as secondary caries that occur in the tooth may progress. Although various studies have been conducted with hydroxyapatite, there is no regenerative and antibacterial commercial material suitable for use in Type 1 and Type 2 restorations since it cannot provide sufficient mechanical properties.

As a result, it is predicted that the production of dental filling materials will provide innovation to minimize problems such as polymerization shrinkage and secondary caries formation for the relevant technical field.

### SUMMARY

The invention relates to a new supportive phase system for the production of light-cured and polymerizable restorative acrylic dental composite filling materials and the said dental composite filling materials in order to eliminate the existing disadvantages in the related technical field and to offer additional technical advantages and solutions for the related technical field.

The main object of the invention is to provide an acrylic dental composite filling material in which edge compatibility is improved by reducing polymerization shrinkage.

Another main object of the invention is to present an acrylic dental composite filling material with improved antibacterial and bioactive properties.

In another aspect, the invention is to provide an acrylic dental composite filling material that minimizes the formation of secondary caries in patients.

The invention is to provide an acrylic dental composite filling material with improved regenerative properties in one aspect.

### DETAILED DESCRIPTION OF THE EMBODIMENTS

In this detailed description, the subject of the invention relates to a new supportive phase system for the production of light-cured and polymerizable restorative acrylic dental composite filling material and the said dental composite filling material and is explained with examples that do not have any limiting effect only for a better understanding of the subject.

The invention relates to the dental filling material to provide all these benefits mentioned for the related technical field. The said dental filling material is a composite material. A composite filling material that can be used as a dental filling material comprises a matrix comprising at least one organic component(s) within the filling material, and a supportive phase system comprising components to provide antibacterial, regenerative, and bioactive properties for the final product.

The supportive phase system within the acrylic dental composite filling material of the invention provides antibacterial properties for the final product and also contains components with high mechanical properties.

The acrylic dental composite filling material of the invention contains biomimetic hydroxyapatite, Al—Sr-OF, and Al—Si—Sr-OF compounds and silica components as the supportive phase. The components included in the supportive phase system of the invention have nanoflower morphology, unlike the present art. The supportive phase system obtained from components with the nanoflower morphology has a high surface area/volume property. In addition, the supportive phase system has a high surface reactivity thanks to the presence of components with these properties. Ultimately, the supportive phase system developed in this way ensures that the performance of the acrylic dental composite filling material to be obtained is increased to very high levels.

The supportive phase system of the invention makes it possible to obtain acrylic dental composite filling material with high antibacterial properties thanks to the Al—Sr-OF and Al—Si—Sr-OF components with nanoflower morphology. These components prevent the formation of secondary caries that can be seen in patients thanks to the fluorides they contain.

The supportive phase system of the invention makes it possible to obtain acrylic dental composite filling material with high biocompatibility and regenerative properties thanks to the biomimetic hydroxyapatite component with nanoflower morphology. In addition to the aforementioned properties, the biomimetic hydroxyapatite component contributes to the improvement of the properties of flexural strength, compressive strength, hardness, curing depth, and polymerization shrinkage for the final product acrylic composite filling material.

In the present invention, the inventors provide methods for the production of each component within the supportive phase in nanoflower morphology.

The supportive phase system of the invention is in the range of 50% to 90% by weight in the dental composite filling material.

The inventors can use three different methods for the production of the biomimetic hydroxyapatite component within the supportive phase system. Microwave irradiation, sonochemical and hydrothermal synthesis are used as the said methods. Under this heading, detailed explanations are made for the production of the hydroxyapatite component in the nanoflower morphology with the said production methods.

### Synthesis of Hydroxyapatite Compound in Nanoflower Morphology

(NH₄)₂HPO₄ and Ca(NO₃)₂·4H₂O solutions are used as raw materials for the production of the hydroxyapatite compound. In addition, as known in the art, synthetic body fluid with composition is used. Accordingly, in nanoflower morphology, the hydroxyapatite component first comprises the following process steps:
i. 50 ml of Ca(NO₃)₂·4H₂O and 0.1 M EDTA mixture in the range of 0.05 to 0.15 M is added to 50 ml (NH₄)₂HPO₄ solution in the range of 0.03 to 0.08 M.
ii. NaOH is added to the solutions taken into SVS and the pH value is increased to 9-13 in a controlled manner and mixed for a few minutes.

Different methods can be applied by the inventors for obtaining biomimetic hydroxyapatite in the solution nanoflower morphology obtained after the application of the process steps i) and ii).

### Obtaining Hydroxyapatite in Nanoflower Morphology by Applying Microwave Method

iii. The solution obtained in the process step ii is treated in a microwave oven so that it is open for at least 6 hours and closed for at least 10 hours;
   preferably, an oven of 700 W power is used as the said microwave.
iv. The mixture obtained by applying process step iii is cooled to room temperature and washed with deionized water.
v. The mixture obtained by applying the process step iv is dried for at least 2 hours in a vacuum oven with a temperature of at least 70° C.

### Obtaining Hydroxyapatite in Nanoflower Morphology by Applying Sonochemical Method

iii. The solution obtained in process step ii is placed in the sonicator device and the mixing process is applied;
   the said sonicator device preferably has a frequency of at least 28 kHz and power characteristics of 200 W.
iv. The mixture obtained in process step iii is exposed to ultrasound energy for at least 90 minutes.
v. Subsequently, hydroxyapatite compounds with the obtained nanoflower form morphology are washed with deionized water and/or ethanol.
vi. It is then subjected to drying processes:
   said drying process is preferably carried out for 24 hours and at a temperature of at least 600° C.

### Obtaining Hydroxyapatite in Nanoflower Morphology by Applying Hydrothermal Method

iii. The mixture obtained in process step ii is subjected to the mixing process before being placed in the hydrothermal reactor;
   the said mixing process is preferably carried out at a speed of at least 300 rpm for 10 minutes.
iv. The mixture obtained in process step iii is placed in the hydrothermal reactor and the hydrothermal reaction is carried out;
   the said hydrothermal reaction is carried out for at least 12 hours and in the temperature range of 150 to 220° C.
v. The mixture is then expected to drop to room temperature.
vi. The precipitate removed from the reactor is rinsed with distilled water.
vii. It is dried in an oven at 60° C. for 24 hours.

The mentioned production methods provide hydroxyapatite compounds in nanoflower morphology. Subsequently, hydroxyapatite compounds are used as components in the supportive phase system in the nanoflower morphology.

### Al—Sr-OF Synthesis in Nanoflower Morphology

The supportive phase system of the invention comprises Al—Sr-OF and Al—Si—Sr-OF compounds as fluorine release agents. These supportive phase systems, which are generally obtained by the melting method, are limited in use as supportive phase systems since they have a large grain size. Al—Sr-OF and Al—Si—Sr-OF metaloxyfluorides with nanoflower morphology produced by the inventors in the invention have been used as supportive phase systems together with hydroxyapatite in nanoflower morphology thanks to their superior mechanical properties. In nanoflower morphology, Al—Sr-OF and Al—Si—Sr-OF compounds can be produced by three different methods similar to the production of hydroxyapatite compounds. For the application of the said production methods, preliminary preparation processes are applied for the production of Al—Sr-OF and Al—Si—Sr-OF compounds. The said process steps are as follows:
i. The cation solution is prepared by mixing 80 mL of Al(NO₃)·9H₂O in the range of 0.1 to 0.3 M, 20 mL Sr(NO₃)₂ in the range of, 0.1 to 0.3 M, and 0.1 M EDTA solutions.
ii. The anion solution is prepared by mixing 720 mL NH₄OH in the range of 0.1 M to 0.3 M and 180 mL NH₄F in the range of 0.1 to 0.3 M.
iii. The cation solution is added to the anion solution under strong stirring.

The synthesis of Al—Sr—Of compounds in nanoflower morphology is possible by applying two different production methods, sonochemical and hydrothermal synthesis methods, to the mixture obtained by applying process steps i-iii.

### Obtaining Al—Sr-OF in Nanoflower Morphology by Applying Sonochemical Method

The solution obtained by the application of process step iii is subjected to stirring for at least 90 minutes at room temperature in the ultrasonic sonicator device, followed by the synthesis of Al—Sr—OF compounds in the nanoflower morphology. It is preferred that the said ultrasonic sonicator device is at least 28 KHz frequency and 200 W power.

### Obtaining Al—Sr-OF in Nanoflower Morphology by Applying Hydrothermal Synthesis Method

The mixture obtained in process step iii is placed in the hydrothermal reactor and the hydrothermal reaction is carried out. The said hydrothermal reaction is carried out for at least 12 hours and at a value in the temperature range of 150 to 220° C.

Al—Si—Sr-OF Synthesis with Nanoflower Morphology
i. The cation solution is prepared by mixing 60 to 70 mL of Al(NO₃)₃·9H₂O in the range of 0.1 to 0.3 M and 30 to 40 mL of Sr(NO₃)₂ and 0.1 M EDTA solutions in the range of 0.1 to 0.3 M.
ii. The anion solution is prepared by mixing 60 to 70 mL of Na₂SiO₃ solution in the range of 0.1 to 0.3 M, 600 to 700 mL of NH₄OH solution in the range of 0.1 to 0.3 M, and 2M 180 mL NH₄F solutions.

The synthesis of Al—Si—Sr—Of compounds in nanoflower morphology is possible by applying two different production methods, sonochemical and hydrothermal synthesis methods, to the mixture obtained by applying process steps i-ii.

### Obtaining Al—Si—Sr-OF in Nanoflower Morphology by Applying Sonochemical Method

The solution obtained by the application of the process step ii is subjected to stirring for at least 90 minutes at room temperature in the ultrasonic sonicator device, followed by the synthesis of Al—Si—Sr-OF compounds in the nanoflower morphology. It is preferred that the said ultrasonic sonicator device is at least 28 kHz frequency and 200 W power.

### Obtaining Al—Sr-OF in Nanoflower Morphology by Applying Hydrothermal Synthesis Method

The mixture obtained in process step ii is placed in the hydrothermal reactor and the hydrothermal reaction is carried out. The said hydrothermal reaction is carried out for at least 12 hours and at a value in the temperature range of 150 to 220° C.

### Silica Synthesis

Silica powders were obtained in the rotary evaporator using Ludox HS-40, a colloidal silica solution for commercial use in the supportive phase system. The experimental stages carried out are outlined below:
i. 100 mL of colloidal silica solution is placed in the 250 mL glass chamber of the rotary evaporator,
ii. During the drying process, the temperature is gradually reduced from 160° C. to 40° C.,
iii. After the temperature is fixed at 40° C., drying continues for 3 hours,
iv. Dried silica powders are mechanically ground with the help of a ball mill for 24 hours,
v. The ground powders are passed through a 250 mesh sieve and used as a supportive phase.

The supportive phase system of the invention is preferably subjected to silanization processes and the silanized supportive phase system is allowed to be obtained. It is a preferred embodiment of the invention that the supportive phase components powders obtained by the production methods given in the invention are combined and subjected to silanization processes.

### Silanization of Supportive Phase Systems

Silanization of the supportive phase system is performed in the nitrogen atmosphere by following the steps below, respectively.
i. In a sealed glass bottle, 5% to 10% by weight of 3methacryloyloxy-propyl-trimethoxysilane is added to the ethanol:water solution in the ratio of 4:1 to 10:1 by weight, and the pH of the solution is adjusted to a value in the range of 3 to 4 pH with the acetic acid solution and mixed at room temperature for 1 hour.
ii. The supportive phase system to be modified under strong stirring is then added to this solution and stirred at room temperature for 24 hours.
iii. After the silane inoculation, the reaction mixture is filtered and rinsed with ethanol to remove the physically adsorbed silanes.
iv. After this process, it is allowed to dry at 60° C. to increase the concentration of silanol molecules and to remove the remaining solvent.

The dental filling material of the invention is a composite material and contains at least one supportive phase and matrix component. The components used as matrix components in the invention are entirely obtained from organic compounds.

### Synthesis of Organic Matrix

The experimental steps followed during the preparation of CQ, TPO, and 4-ethyl dimethylaminobenzoate (4-EDMAB) components used as organic matrix and photoinitiator performed in the specified step are described below.
i. Preheating is performed by placing BisGMA and TEGDMA in the oven at 37° C.
ii. BisGMA and TEGDMA are weighed with precision scales and placed in the container of the Speedmixer for mixing.
iii. Photoinitiators named CQ, TPO, and 4-EDMAB are weighed with precision scales to adhere to their compositional ratios so that the dental composite to be produced can be cured with a blue light photocuring device.
iv. Organic phases and photoinitiators are placed in the same container and subjected to strong stirring 3 times at 2000 rpm in 5-minute periods.

### Synthesis of Acrylic Dental Composite Filling Material

i. BisGMA in the range of 1% to 5% by weight is stirred at a constant temperature for 10 minutes in the ultrasonic water bath,
ii. The organic resin part of the composite structure is prepared by adding TEGDMA in the range of 1%-5% by weight to the mixture obtained in the process step i),
iii. Camphorquinone in the range of 0.05% to 0.2% by weight and Diphenyl(2,4,6-trimethylbenzoyl) phosphine oxide compound in the range of 0.05% to 0.2% by weight are added to the organic resin mixture obtained in the process step ii) as photoinitiator and 4-EDMAB compound in the range of 0.5% to 1% by weight as an activator and then the mixture is heated for 10 minutes,
iv. The supportive phase system in the ratio of 50-90% by weight to the organic resin mixture obtained in the process step iii) and the composite filling materials are obtained as a result of the mixing process in the ultrasonic water bath or by means of a speed mixer for at least one day until a homogeneous mixture is obtained,
   the said supportive phase system comprises the hydroxyapatite compound with nanoflower morphology, the Al—Si—Sr-OF compound with nanoflower morphology, the Al—Sr-OF compound with nanoflower morphology, and the silica compound,
v. The composite filling materials obtained in process step iv are placed in the teflon molds with the spatula by bringing them to room temperature, then curing processes are applied using a blue-LED light device.

The use of hydroxyapatite, Al—Sr-OF, and Al—Si—Sr-OF supportive phase systems with nanoflower morphology in restorative dental composites is new in the relevant technical field, separately and together. Thus, it is possible to obtain a composite filling material with high biocompatibility, antibacterial and improved mechanical properties compared to the dental composites in the present art. Nanodimensional supportive phase systems with high surface reactivity and surface area/volume ratio are obtained with nanoflower morphology obtained with more sensitive process steps compared to the current methods. As a result, regenerative and antibacterial properties of composite filling material are increased with hydroxyapatite, Al—Sr-OF and Al—Si—Sr-OF supportive phase systems with nanoflower morphology, while improving their mechanical properties. Thus, the mechanical, physical, chemical and antibacterial properties of dental composite filling materials containing BHA, Al—Sr-OF and Al—Si—Sr-OF supportive phases with nanoflower morphology formed by the combination of nanosized rods in a global form can be simultaneously optimized. With this application, the minerals and elements in the original structure of the tooth can be synthesized to have nanoflower morphology and added to the dental composite structure. By overcoming the problem of low mechanical strength, which is the biggest obstacle to this situation until today, different and more advantageous filling materials can be produced from the current solution proposals.

The scope of protection of the invention is specified in the attached claims and cannot be limited to those explained for sampling purposes in this detailed description. It is evident that a person skilled in the art may exhibit similar embodiments in light of the above-mentioned facts without drifting apart from the main theme of the invention.

What is claimed is:

1. An acrylic dental composite filling material comprising a light-cured and polymerizable organic compound, a photoinitiator, and a supportive phase system, wherein the acrylic dental composite filling material comprises:
a hydroxyapatite component with a nanoflower morphology to provide regenerative properties to the acrylic dental composite filling material within the supportive phase system,
compounds comprising Al—Si—Sr-OF and Al—Sr-OF fluorides having nanoflower morphologies as the supportive phase system to give antibacterial properties to the acrylic dental composite filling material within the supportive phase system,
a silica component within the supportive phase system,
a mixture of bisphenol A-glycidyl methacrylate (BisGMA) and triethylene glycol dimethacrylate (TEGDMA) as the light-cured and polymerizable organic compound,
at least one of the group consisting of camphorquinone (CQ), diphenyl(2,4,6-trimethylbenzoyl) phosphine oxide, 1-phenyl-1,2-propanedione, and 4-ethyl dimethylaminobenzoate (4-EDMAB) as the photoinitiator.

2. The acrylic dental composite filling material according to claim 1, wherein the BisGMA is in a range of 1%-5% by weight.

3. The acrylic dental composite filling material according to claim 1, wherein the TEGDMA is in a range of 1%-5% by weight.

4. The acrylic dental composite filling material according to claim 1, wherein the CQ is in a range of 0.1%-0.5% by weight.

5. The acrylic dental composite filling material according to claim 1, wherein the diphenyl(2,4,6-trimethylbenzoyl) phosphine oxide is in a range of 0.1%-0.5% by weight.

6. The acrylic dental composite filling material according to claim 1, wherein the 1-phenyl-1,2-propanedione is in a range of 0.1%-0.5% by weight.

7. The acrylic dental composite filling material according to claim 1, wherein the 4-EDMAB is in a range of 0.5%-1.0% by weight.

8. The acrylic dental composite filling material according to claim 1, wherein an amount of the supportive phase system is in a range of 50%-90% by weight.

9. The acrylic dental composite filling material according to claim 1, further comprising a pigment in addition to the light-cured and polymerizable organic compound and the supportive phase system.

10. The acrylic dental composite filling material according to claim 9, wherein the pigment is in a range of 0.01%-1% by weight.

11. The acrylic dental composite filling material according to claim 9, wherein the pigment is at least one selected from the group consisting of Duranat Yellow Iron Oxide (Pigment Yellow 42 & 43 CI 77492), Duranat Red Iron Oxide (Pigment Red 101 CI 77491), phenylbis(2,4,6-trimethylbenzoyl) phosphine oxide, 1-phenyl-1,2-propanedione 98%, the diphenyl(2,4,6-trimethylbenzoyl)phosphine oxide, Duranat Brown Iron Oxide (Pigment Brown), Duranat Black Iron Oxide (Pigment Black 11 CI 77499), iron oxide (Fe₂O₃—red), ferric hydroxide (FeOOH—yellow), TiO₂, E171 Titanium Dioxide, and Pigment White 6 CI 77891.

12. A method of producing the acrylic dental composite filling material according to claim 1, comprising the following process steps:
i. stirring the BisGMA in a range of 1% to 5% by weight at a constant temperature in an ultrasonic water bath to obtain a first mixture,
ii. adding the TEGDMA in a range of 1%-5% by weight to the first mixture obtained in the process step i) to prepare an organic resin mixture,
iii. adding the CQ in a range of 0.05% to 0.2% by weight and the diphenyl(2,4,6-trimethylbenzoyl) phosphine oxide in a range of 0.05% to 0.2% by weight as the photoinitiator and the 4-EDMAB in a range of 0.5% to 1% by weight as an activator to the organic resin mixture prepared in the process step ii) to obtain a second mixture, and heating the second mixture is for 10 minutes,
iv. adding the supportive phase system in a ratio of 50% to 90% by weight to the second mixture after heating to obtain a third mixture, subjecting the third mixture to a mixing process in the ultrasonic water bath or by means of a speed mixer until a homogeneous mixture is obtained, wherein the homogeneous mixture is the acrylic dental composite filling material;
the supportive phase system comprises the hydroxyapatite component with the nanoflower morphology, the Al—Si—Sr-OF compound with the nanoflower morphology, the Al—Sr-OF compound with the nanoflower morphology, and the silica compound,
v. placing the acrylic dental composite filling material obtained in the process step iv) in a teflon mold with a spatula by bringing the acrylic dental composite filling material to room temperature,
vi. applying a curing process by using a blue-LED light device.

13. The method of producing the acrylic dental composite filling material according to claim 12, wherein a mixing process is carried out at 40° C. for 10 minutes in the process step i).

14. The method of producing the acrylic dental composite filling material according to claim 12, wherein the supportive phase system is added in the ratio of 70% by weight in the process step iv).

15. The method of producing the acrylic dental composite filling material according to claim 12, wherein a mixing process is carried out for 3 hours in the process step iii).

16. The method of producing the acrylic dental composite filling material according to claim 12, wherein the mixing process is carried out for 1 day in the process step iv).

17. The method of producing the acrylic dental composite filling material according to claim 12, wherein the curing process with the blue-LED light device is carried out for 20 seconds in the process step vi).

18. The acrylic dental composite filling material according to claim 2, further comprising a pigment in addition to the light-cured and polymerizable organic compound and the supportive phase system.

19. The acrylic dental composite filling material according to claim 3, further comprising a pigment in addition to the light-cured and polymerizable organic compound and the supportive phase system.

20. The acrylic dental composite filling material according to claim 4, further comprising a pigment in addition to the light-cured and polymerizable organic compound and the supportive phase system.
